(19) United States
(12) Patent Application Publication
(10) Pub. No.: US 2025/0387147 A1
(43) Pub. Date: Dec. 25, 2025

(54) INTRAMEDULLARY NAIL, KIT, AND METHOD

(71) Applicant: Medline Industries, LP, Northfield, IL (US)

(72) Inventors: Ryan Niver, Glenview, IL (US); Samuel Nader, Arlington Heights, IL (US); Dinesh Koka, Winter Park, FL (US); Natan Pheil, Highland Park, IL (US)

(21) Appl. No.: 19/245,907

(22) Filed: Jun. 23, 2025

Related U.S. Application Data
(60) Provisional application No. 63/663,899, filed on Jun. 25, 2024.

(51) Int. Cl. A61B 17/72; A61B 17/86; A61B 17/88; A61B 17/90

(52) U.S. Cl. CPC A61B 17/7233; A61B 17/86; A61B 17/8872; A61B 17/90

(57) ABSTRACT

Disclosed is an intramedullary nail useful in the repair of fibular fractures. The nail has a proximal portion with a threaded outer surface and a threaded distal portion. The distal portion may have a tapered or partially tapered configuration or a staggered configuration. The nail may be used with a placement jig that has holes aligned with transverse holes in the nail to enable placement of securement screws and other devices. An associated kit and method are also disclosed.

### FIELD

This invention is in the field of orthopedic implants. Generally, the disclosure relates to an intramedullary nail and associated kits and methods.

### BACKGROUND

Intramedullary nails are known for use in the fixation of fibula fractures and osteotomies, among other uses. In a fibula fracture repair, an intramedullary nail is inserted into the intramedullary fibular cavity and secured via transverse screws to other bone structures, either via trans-syndesmotic, infra-syndesmotic, or supra syndesmotic connections. The present disclosure seeks to provide an intramedullary nail for fibular repair that provides enhanced fixation and optimally enhanced flexibility to accommodate the curvature in the fibular cavity.

Now disclosed is an intramedullary nail that includes an elongate body having a distal portion and a proximal portion, wherein the proximal portion comprises an outer surface having a threaded area, generally a non-compressing threaded area, and wherein the distal portion comprises a helical thread formed thereon. The helical thread in the distal area has a major diameter and a non-uniform minor diameter region, which may be, for instance, a tapered or staggered minor diameter region. The threaded proximal portion allows for securement of the nail relative to the fibula or other bone of patient during installation. The non-uniform minor diameter is intended to allow for flexibility of the distal end of the implant.

Also disclosed are embodiments of a placement jig. The placement jig has openings aligned with transverse holes in the intramedullary nail to align and register securement screws or other securement devices to assist in securement of the intramedullary nail.

Also described herein is a kit that includes the intramedullary nail and associated components, such as one or more of a placement jig, transverse securement screws, a drive assembly, and other components. Further disclosed herein is a method for implanting an intramedullary nail as described herein, the method including forming an appropriately sized channel in an intramedullary cavity, securing the intramedullary nail therewithin via the threaded area of the proximal portion, and securing the nail using one or more securing screws or other fasteners.

### BRIEF DESCRIPTION OF THE DRAWINGS

FIGS. 1-3 are side elevations of exemplary intramedullary nails in accordance with the present disclosure.

FIG. 4 is a relatively enlarged view of the distal portion of the intramedullary nail shown in FIG. 1.

FIG. 5 is a relatively enlarged side elevation of the distal portion of a first alternative intramedullary nail.

FIG. 6 is a relatively enlarged side elevation of the distal portion of a second alternative intramedullary nail.

FIG. 7 is a perspective view illustrating a front quarter of the intramedullary nail shown in FIG. 1

FIG. 8 is a perspective view of a jig useful in placing the intramedullary nail shown in FIG. 1.

FIGS. 9 and 10 are top and rear views respectively of the jig shown in FIG. 8.

FIG. 11 is a perspective view of a transverse securement screw.

FIG. 12 is a perspective view of a portion of the proximal end of the intramedullary nail shown in FIG. 1, shown with two transverse distal securement screws and two syndesmotic securement screws installed.

FIG. 13 is a perspective view of a patient's bone structure showing the intramedullary nail installed and including transverse distal securement screws and syndesmotic securement screws.

FIG. 14 is a front elevation of the bone structure shown in FIG. 13.

FIG. 15 is a representation of a kit that includes an intramedullary nail, plural securement screws, a jig, a guide wire, and a reamer.

FIG. 16 is a bottom plan view of a jig assembly including the jig placed over a driver and locking rod.

FIG. 17 is a bottom-oriented perspective view of an end of the jig assembly shown in FIG. 16

FIG. 18 is a top-oriented perspective view of the jig assembly shown in FIGS. 16 and 17.

FIG. 19 is a relatively enlarged view of the end of the driver and connecting rod of the jig assembly shown in FIG. 18

FIG. 20 is an exploded view of the jig assembly shown in FIG. 18, shown further with drill and screw guide sleeves, and including a callout showing a relatively enlarged view of the spring bearing assembly.

FIG. 21 is a relatively enlarged perspective view of certain components of the jig shown in FIG. 18, with the locking knob of the jig shown in hidden lines.

FIG. 22 is a perspective view of an assembly of the intramedullary nail shown in FIG. 1 with a driver, the driver shown in hidden lines.

FIGS. 23-32 illustrate steps in a method of implanting the intramedullary nail shown in FIG. 1 using the jig assembly and other components shown in prior Figures.

FIGS. 33 and 34 are side elevations of another embodiment of an intramedullary nail.

FIG. 35 is a perspective view of an alternative jig useful in the placement of the intramedullary nail shown in FIGS. 33 and 34.

FIGS. 36 and 37 are respectively front and rear elevations of the jig shown in FIG. 35.

FIG. 38 is a top elevation of the jig shown in FIG. 35.

FIGS. 39 and 40 are first and second exploded views of the major components of the jig shown in FIG. 35.

FIGS. 40A and 40B are side views of a screw depth gauge useful in a method of placement of the intramedullary nail, and FIG. 40C is a side view of a drill useful in placement of securement screws.

FIGS. 41-50 are views illustrating sequential steps in the placement of the intramedullary nail of claim 35 into the fibula of a patient.

FIG. 51 is an elevation of the jig shown in FIG. 35 with the outrigger section positioned for a right fibula repair.

Terms of orientation uses herein are used for convenient reference to the drawings. In practice, the items depicted may be oriented omnidirectionally.

### DETAILED DESCRIPTION

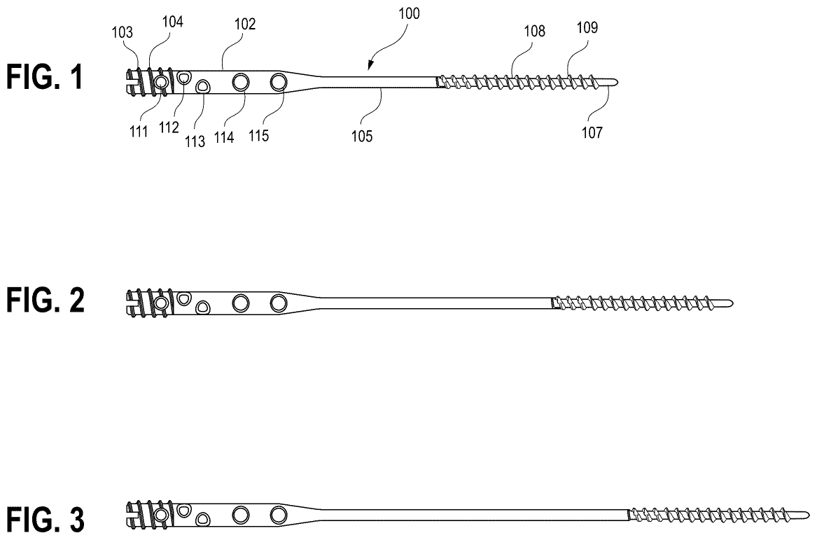

The intramedullary nails shown in FIGS. 1-3 are generally similar in construction although of different shaft lengths. With reference to FIG. 1, the intramedullary nail 100 includes a proximal portion 102 having an outer surface 103 that is threaded via one or more threads. In the illustrated embodiment, a single thread 104 is included. The intramedullary nail includes a shaft 105 connecting the proximal portion to a distal portion 107, the distal portion comprising a threaded area 108 again comprising one or more threads. As shown, a single thread 109 is provided. The terms “proximal” and “distal” herein are used with reference to the hereinabove identified portions of the nail. In use, the distal end of the nail is intended to be positioned relatively closer to the head of the patient and the proximal end of the nail is intended to be positioned relatively further from the head of the patient.

The proximal portion 102 includes a series of transverse through-holes 111, 112, 113, 114, and 115. As seen, the intramedullary nail has a first set of holes 114, 115 positioned for syndesmotic securement, and a second set of holes 111, 112, 113 positioned for transverse non-syndesmotic fibular securement. Each of the first set of holes 114, 115 is spaced apart along an axis of the intramedullary nail. Also, each the first set of holes has an axis, where the axes of the first set of holes are generally parallel to one another, as seen in FIGS. 1-3. Each of the second set of holes 111, 112, 113 also is spaced apart along an axis of the intramedullary nail. At least one hole (in this case, hole 111) of the second set of holes 111, 112, 113 may have an axis that is generally parallel to the axes of the first set of holes. At least one hole of the second set of holes (in this case, both holes 112 and 113) has an axis that is not generally parallel to the axes of the first set of holes.

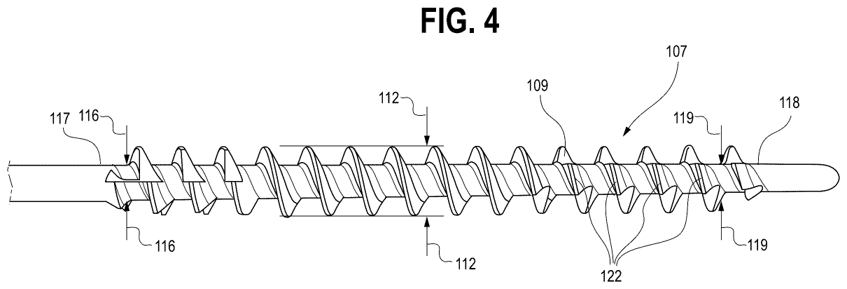

As shown first in FIG. 4, the thread 109 at the distal portion 107 of the intramedullary nail has a major diameter and a non-uniform minor diameter region. The major diameter in the illustrated embodiment is constant across most of the length of the nail and is defined as the smallest cylinder within which the tips of the thread 109 would conceivably fit, as shown via dimension 112. The minor diameter in this embodiment is not uniform in that it tapers from a relatively proximal portion 114, where it is thicker as seen via dimension 116, to a relatively distal portion 118, where it is thinner as seen via dimension 119, which is smaller than dimension 116. In this manner the distal portion 118 is made relatively flexible compared to an otherwise similar intramedullary nail having a uniform minor diameter. Also, as seen, the helical thread 109 is interrupted as seen with flat sections 122.

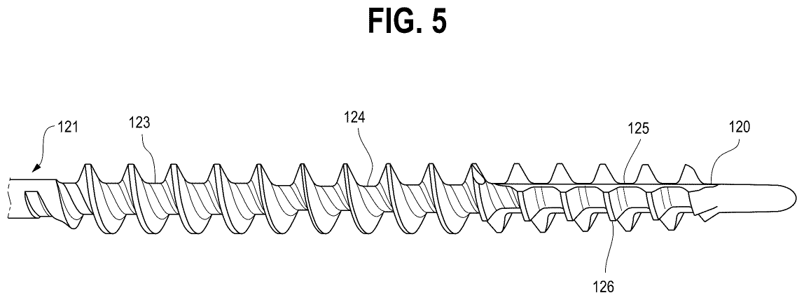

The minor diameters of the nail may have an interrupted configuration. As shown in FIG. 5, the distal end 120 of the alternative intramedullary nail 121 has three zones, a proximal zone 123, an intermediate zone 124, and a distal zone 125. The proximal zone 123 has a minor diameter that is constant, and the distal zone 125 has a smaller minor diameter that is also constant. The intermediate zone 124 has a minor diameter region that is tapered, and the thread 126 is interrupted at in the distal zone 125. The nail 121 is otherwise similarly configured to the nail 100 shown in FIG. 1, except that only a portion of the distal threaded portion of the nail is tapered. In this manner the distal portion 120 is made relatively flexible compared to an otherwise similar implant having a uniform minor diameter. The distal thread of the nail is interrupted with flat sections, as shown.

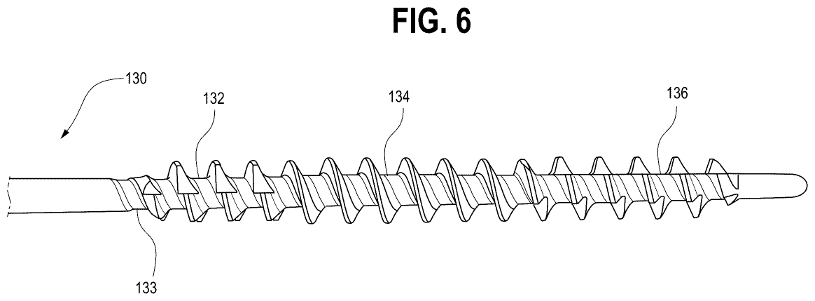

The alternative intramedullary nail 130 in FIG. 6 is likewise similarly configured to the nail 100 shown in FIG. 1 except that the minor diameter region varies in dimension from a relatively thin portion 132 at the most proximal end 133, to a relatively thick intermediate portion 134, and back to a relatively thin distalmost portion 136. In this manner the distal threaded portion of the nail has a staggered configuration, again being non-uniform and flexible relative to an otherwise similar implant having a uniform minor diameter. This nail 130 also has an interrupted helical distal thread with flat sections. In alternative embodiments (not shown) the minor diameter may have plural alternating thicker and thinner regions, separated by abrupt transitions or by tapered regions.

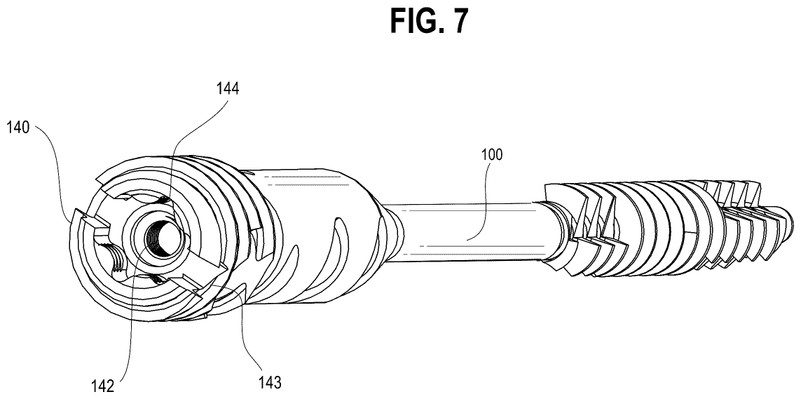

As seen in FIG. 7, the nail 100 includes a head portion 140 having an interior threaded socket 142, and a driver receiving fitting 144, which, in the illustrated embodiment, is a star fitting including a slot 143. The head portions of the alternative intramedullary nails 121, 130 (not shown) are configured similarly.

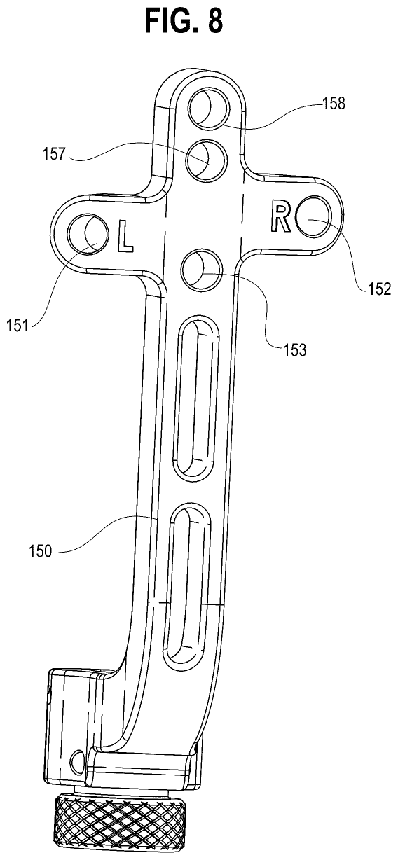

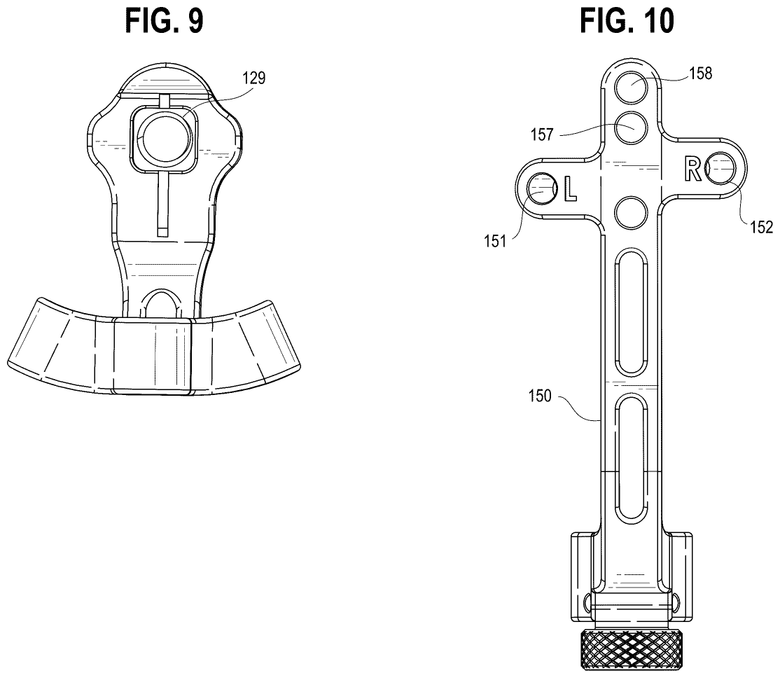

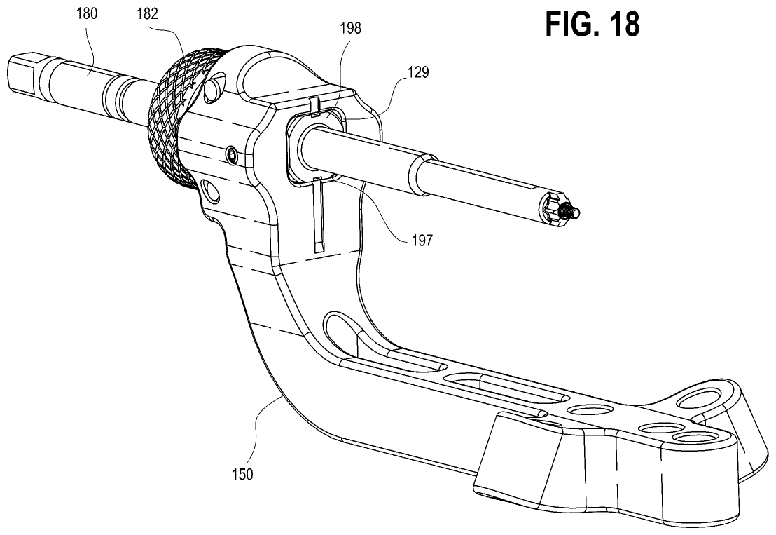

As seen in FIGS. 8-10, the intramedullary nail 100 and the other nails described herein are intended to be used in connection with a placement jig 150, further details of which are discussed below. The placement jig comprises a keyed driver opening 129 (FIG. 9) that cooperates with a keyed portion 198 of a driver 180 (FIG. 18), the driver being accommodated within the keyed driver opening 129 and seating in a socket portion thereof. As described in more detail below, the placement jig includes a plurality of openings, where at least some and preferably all of the openings are positioned for alignment with respective holes in the intramedullary nail when said keyed portion 198 of the driver 180 is positioned within said keyed driver opening 129 and when said driver is in engagement with said nail. The jig should be sized and configured for the particular embodiment of the intramedullary nail.

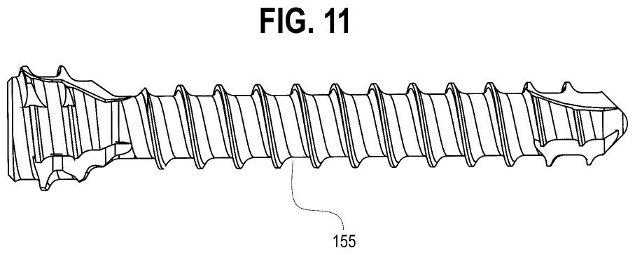

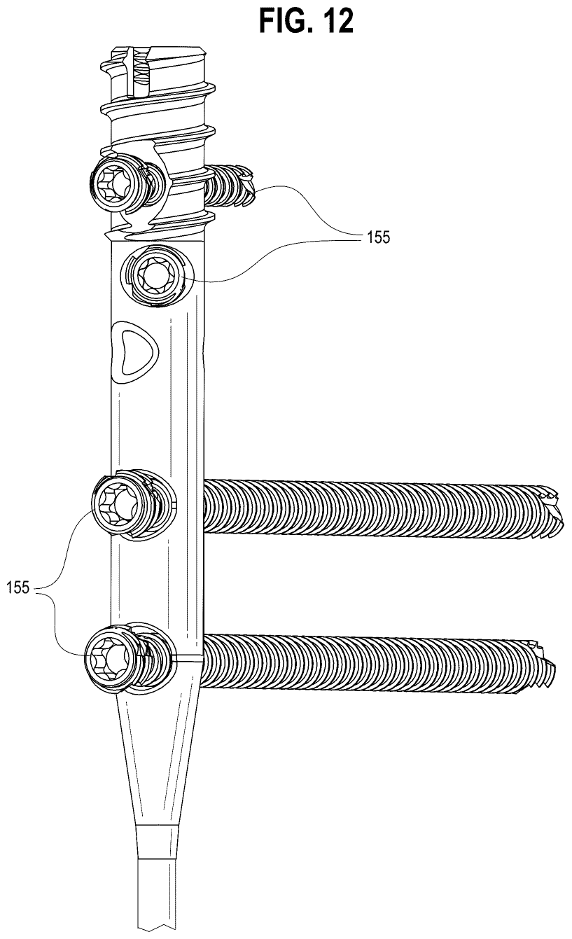

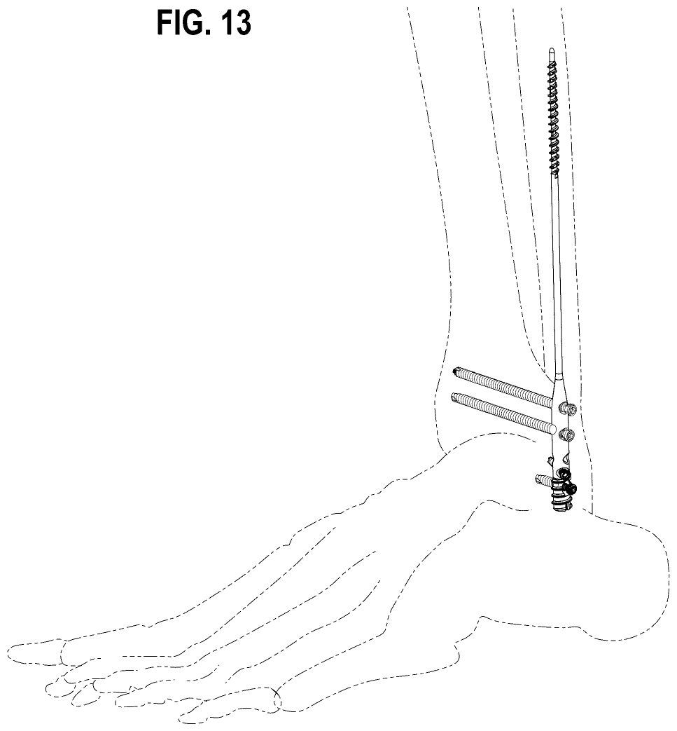

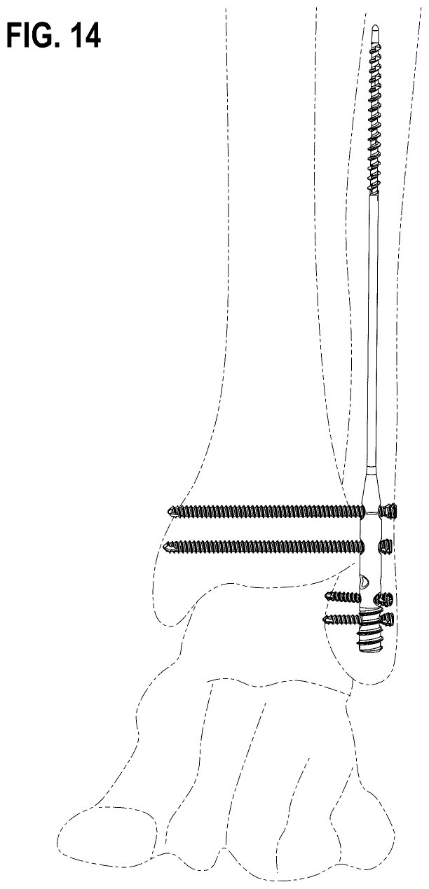

As seen in FIGS. 8 and 10, the illustrated placement jig 150 includes markings “L” and “R” and associated screw passages 151, 152 corresponding to the intended use of the jig on the left or right fibula of a patient. The placement jig further is equipped with central screw passage 153. Each of these are screw passages 151-153 is intended to accommodate a transverse securement screw 155 as seen in FIG. 11. The jig further includes syndesmotic device passages 157, 158 designed to accommodate syndesmotic devices, such as screws 159 as seen in FIG. 12 or alternative syndesmotic securement devices such as buttons (not shown). The placement of the passages 151-153 are 157-158 is intended to align with holes 111-113 and 114-115 respectively when the driver is positioned within the jig and attached to the nail 100. In this manner, the placement jig 150 may be used to secure transverse securement screws 155, and syndesmotic screws 159 or other devices, as seen in FIGS. 13 and 14 with respect to the left foot of a patient.

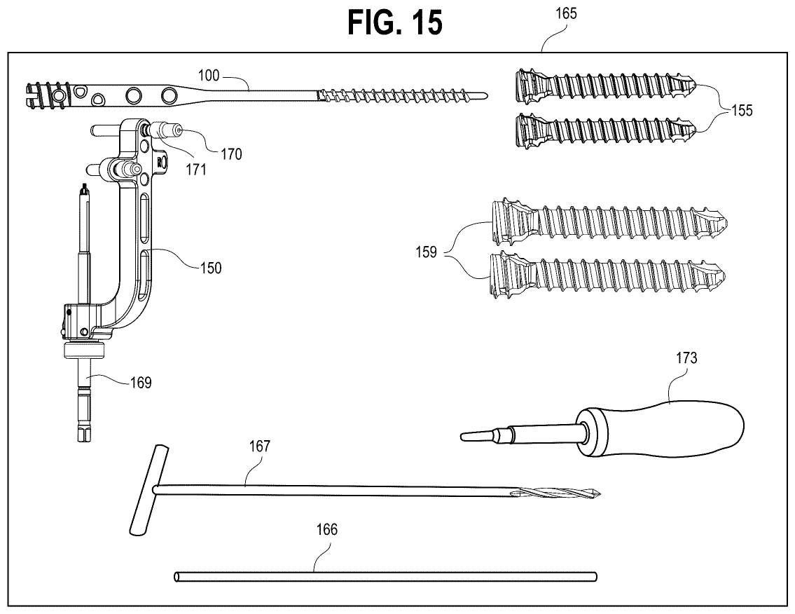

In use, the components described herein possibly are provided in the form of a kit 165, as seen in FIG. 15. Kit 165 includes an intramedullary nail 100, transverse securement screws 155, and syndesmotic screws 159. Optionally, the kit 165 also includes a guide wire 166, a reamer 167, and the placement jig 150. The placement jig 150 as illustrated in FIG. 15 is provided with a drive assembly 169 as hereinafter described and with plural drill sleeves 170 and screw guide sleeves 171, one set of each shown. Additional drill and screw guide sleeves may be provided if desired. The kit may include other components such as a ratchet tool (not shown) for advancing the driver or other suitable components. In practice, a kit may include the nail 100 and one or more securement screws 155 or syndesmotic screws 159 within a container, such kit being sterilely packaged. Other components of the kit may be assembled in situ by an operating team. Because the jig, reamer, guide wire, and other implements described herein are durable and reusable, in many cases these will not be provided in a sterile package but will be sterilized at the surgical facility prior to use. Alternatively, the screws or other fastening devices may be provided separately and not in the form of a sterile packaged kit with the placement jig.

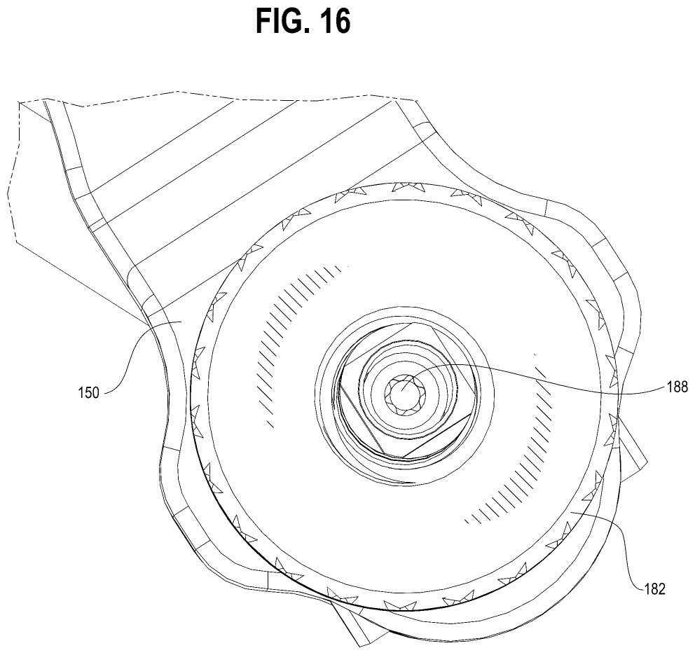

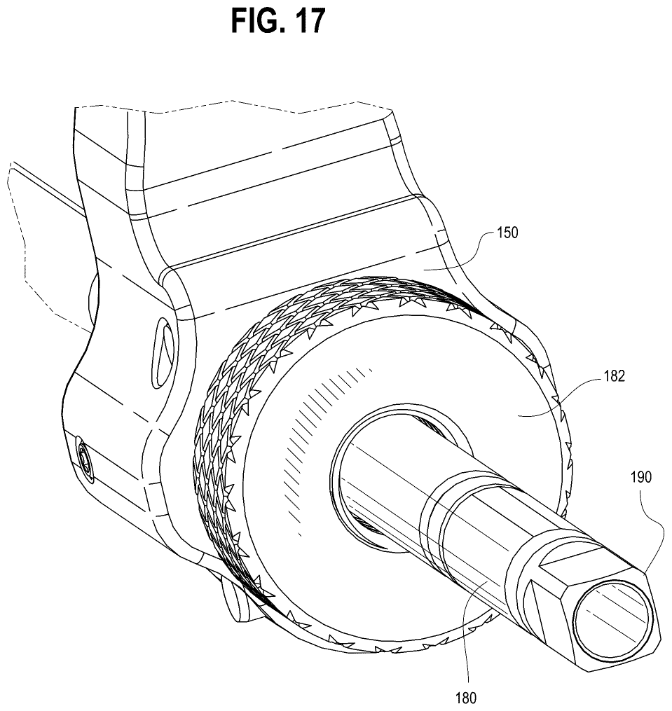

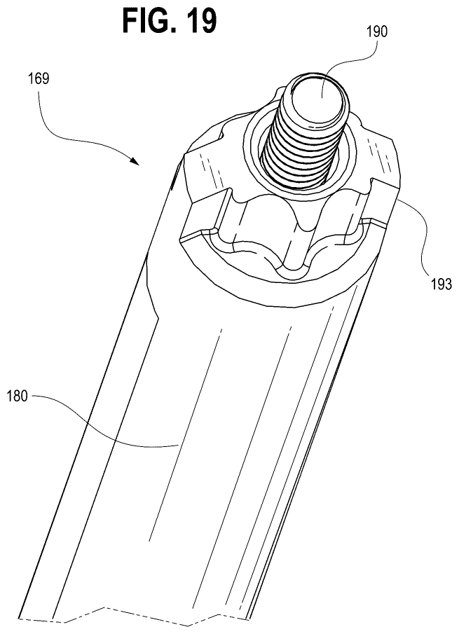

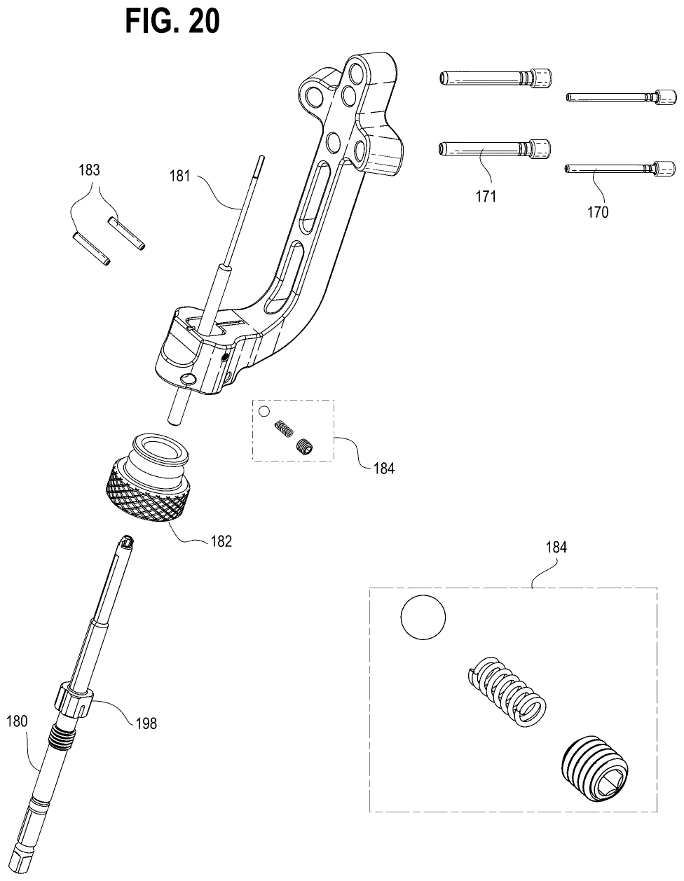

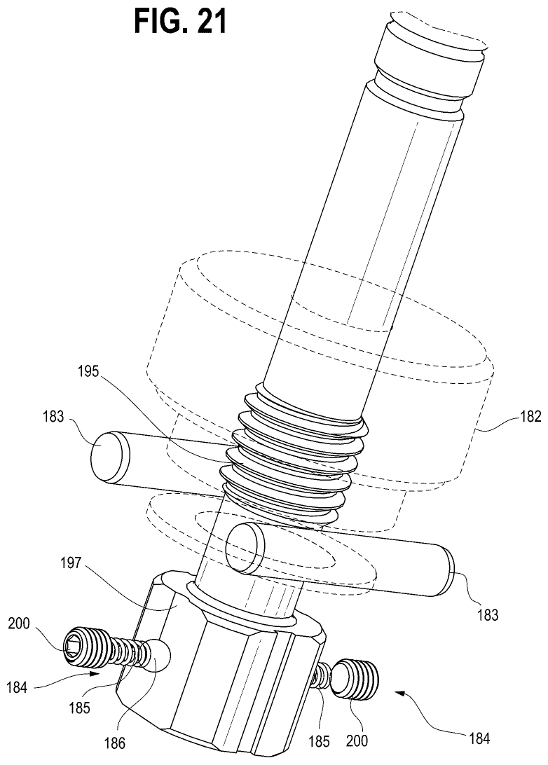

As seen in FIGS. 16-20, the jig and associated components include, with reference first to FIG. 20, a cannulated driver 180 that receiving a connecting rod 181, a locking knob 182, dowel pins 183, drill sleeves 170, screw guide sleeves 171, and two spring bearing assemblies 184. The connecting rod 181 and cannulated driver 180 together form drive assembly 169. As seen in FIG. 16, the head 187 of the connecting rod 181 includes a star drive socket 188 that may be driven by a connecting rod driver. With reference to FIG. 19, the distal end 190 of the connecting rod is threaded and seats within the threaded socket 142 in the head of the intramedullary nail as discussed hereinabove. As seen in FIG. 17, the head 192 of the driver 180 is configured to receive a ratchet tool for urging rotation of the driver, and, returning to FIG. 19, the distal end 193 of the driver has a geometry configured to engage the driver receiving portion 144 in the head 140 of the intramedullary nail.

The drive assembly 169 is fastened to the intramedullary nail 100 for installation. As seen in FIGS. 18 and 21, when the body of the jig (not shown in FIG. 21) is placed over the drive assembly 169, it may be advanced until the keyed portion 198 of the driver engages the keyed driver opening 129. The device is sized such that at this point the locking knob 182 will meet the threaded portion 195 of the driver 180 and may be threaded thereto. The surgeon may then rotate the locking knob to fix the position of the driver relative to the intramedullary nail. The dowel pins 183 capture the locking knob 182 but allow the locking knob to rotate relative to the body of the jig. The jig is further equipped with spring bearing assemblies 184 that cooperate with channels, one shown at 197 in the keyed portion 198 of the driver 180. The spring bearing assemblies 184 include set screws 200 that retain springs 185, the springs 185 urging bearings 186 towards the keyed portion 198. This arrangement helps seat and stabilize the driver 180 in the jig 150 and helps to accommodate tolerances in the sizing of these components.

As seen in FIG. 18, the keyed portion 198 fits within and cooperates with the keyed driver opening 129 in the jig 150 to prevent rotation of the drive assembly 169 when the jig has been placed over the drive assembly 169. Once the locking knob has been secured, neither rotation nor translation of motion of the jig relative to the drive assembly is permitted. Because the connecting rod 181 also connects the drive assembly to an intramedullary nail, the position of the jig is both rotationally and translationally affixed with respect to the nail.

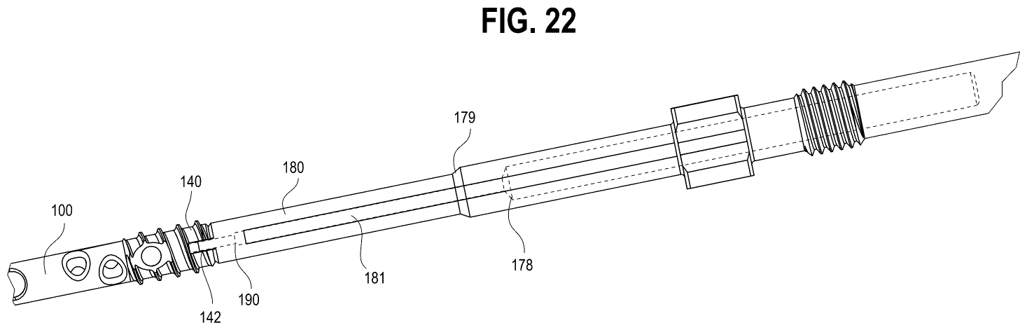

As seen in FIG. 22, the locking rod 181 includes a beveled surface 178 that abuts a bevel 179 in the interior cannula of the driver 180, permitting advancement of the connecting rod 181 to the correct position within the driver 180 and securement of the threaded distal end 190 within the threaded socket 142 of the head of the nail 100.

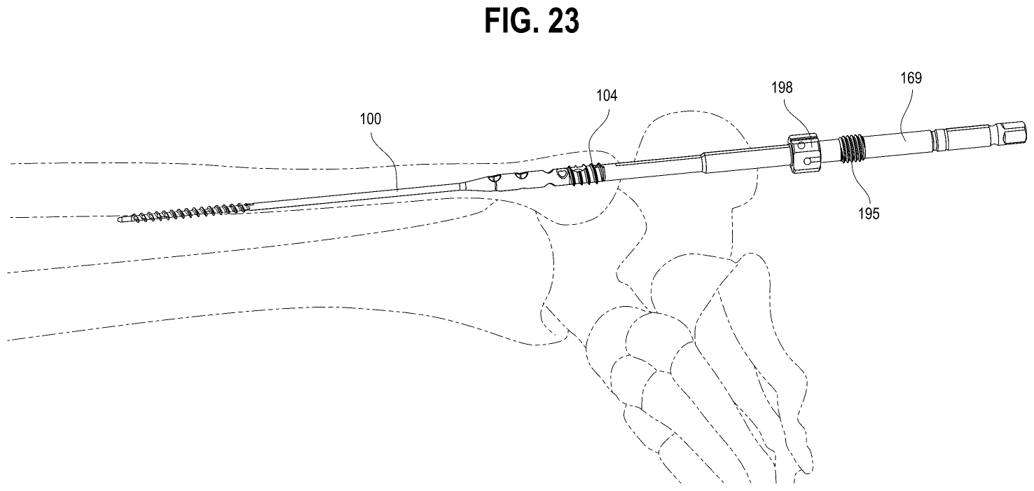

To place the nail, the surgeon first uses the guidewire 166 and reamer 167 to create a channel in the intramedullary cavity, as is conventional. The surgeon then places the cannulated driver 180 into engagement with the nail such that the distal end 193 of the driver engages with the head 140 of the nail. The surgeon then screws the connecting rod 181 into the head of the nail using a suitable tool. As seen in FIGS. 22 and 23, this affixes the nail 100 to the drive assembly 169. A surgeon then may advance the drive assembly 169 to thereby drive the nail forward using a ratchet tool (not shown) or other suitable tool. Advantageously, the thread 104 engages bone within in the intramedullary cavity and is useful in stabilizing the fibula while stabilizing screws are placed, and to address potential movement of the fibula during screw placement. Because both proximal and distal ends of the intramedullary nail are threaded, both proximal and distal portions of the intramedullary nail will be threadingly engaged with bone.

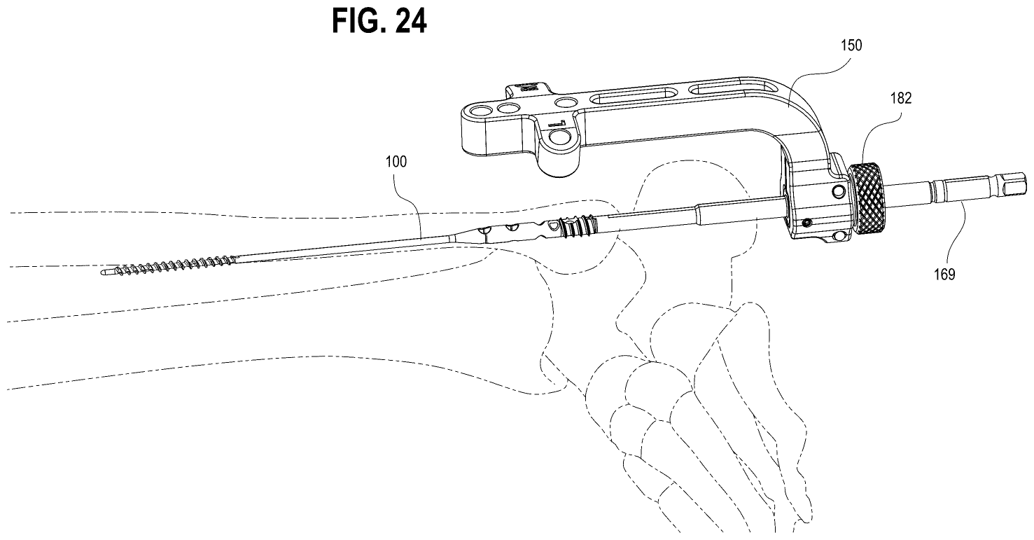

As seen now in FIG. 24, with the drive assembly 169 affixed to the nail 100, the jig 150 is then placed over the drive assembly and the locking knob 182 is then threaded onto the threaded portion 195 (not seen in FIG. 24) of the driver, wherein the keyed portion 198 of the driver seats in the socket defined by the keyed driver opening 129. The driver may be provided with indexing markings (not shown) to assist the surgeon in ensuring proper orientation. The keyed portion 198 and keyed driver opening 199 in the jig 150 ensure that the driver can only fit in the proper orientation or 180 degrees opposing the proper orientation. This configuration is intended to allow for flexibility in positioning of the jig.

The surgeon then typically will use radiographic techniques to ensure that the nail is correctly radially oriented in a desired position relative to the patient's bone structure prior to placing the jig. Because the drive assembly 169 is fixed relative to the nail 100, rotating the drive assembly 169 will cause nail 100 to rotate.

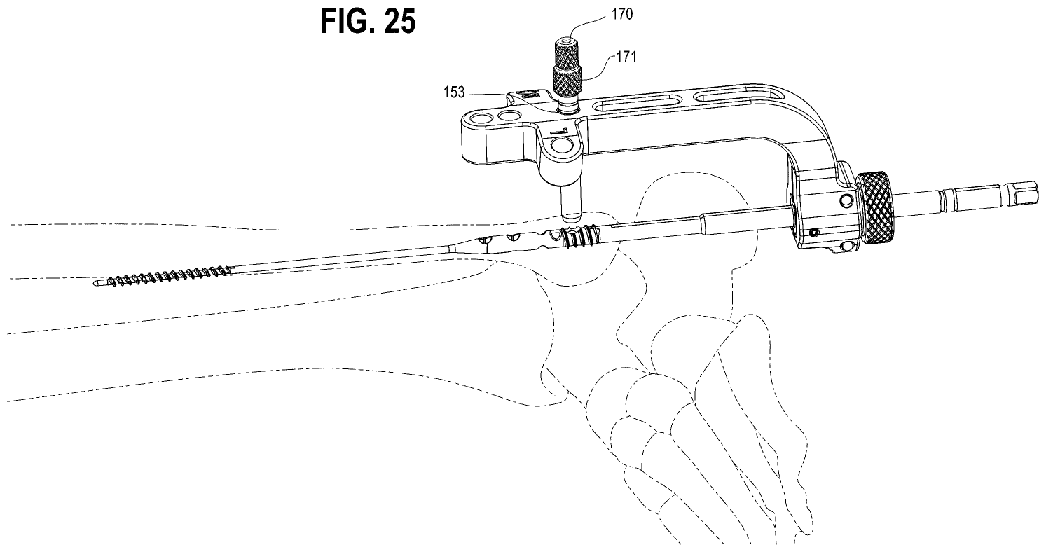

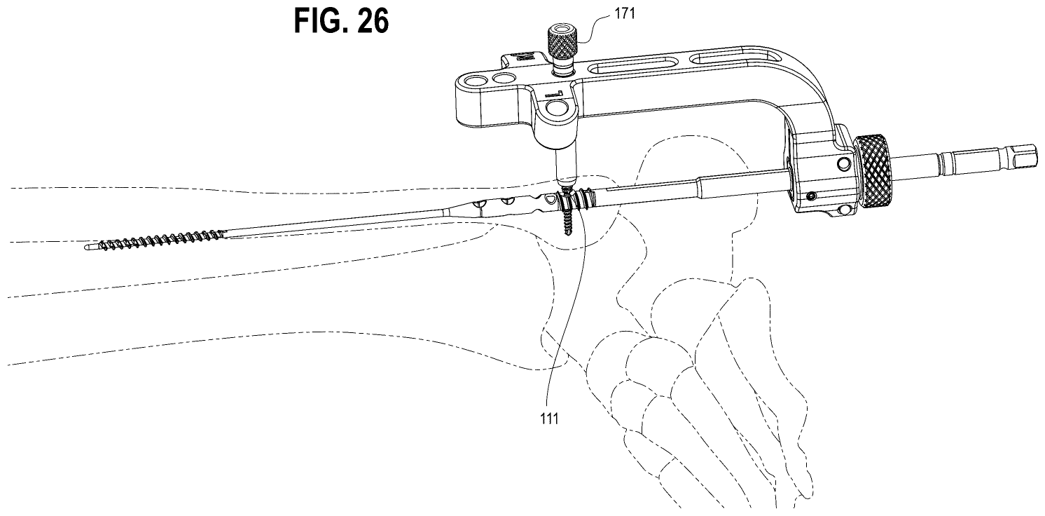

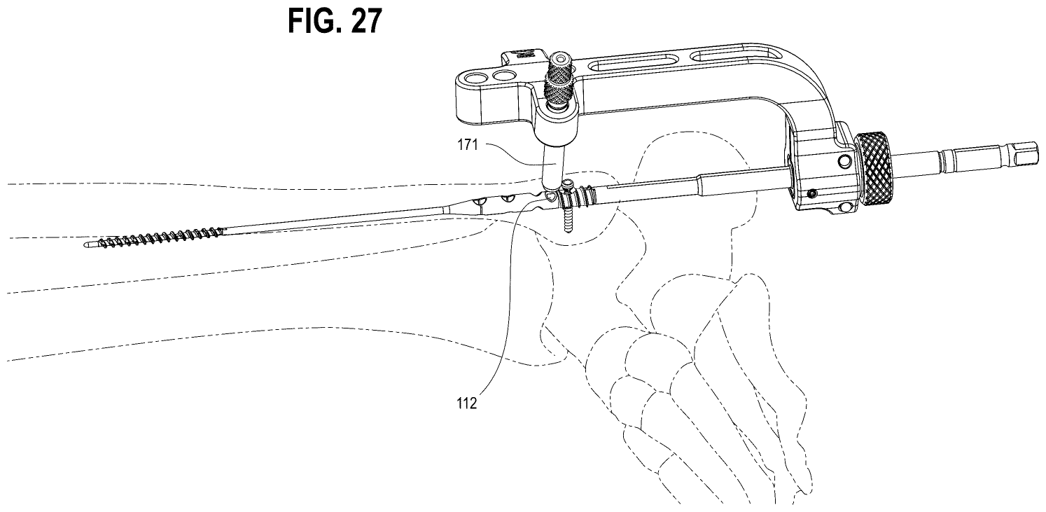

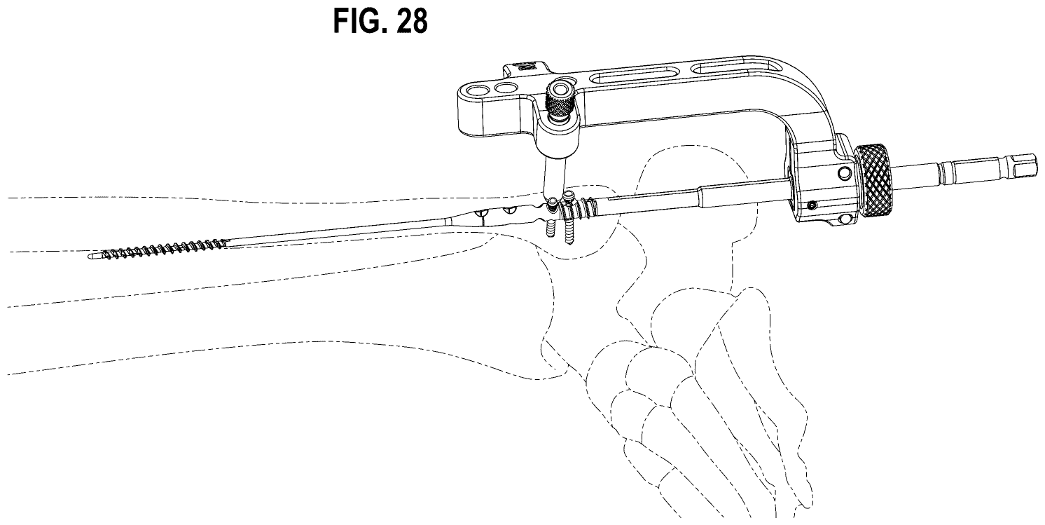

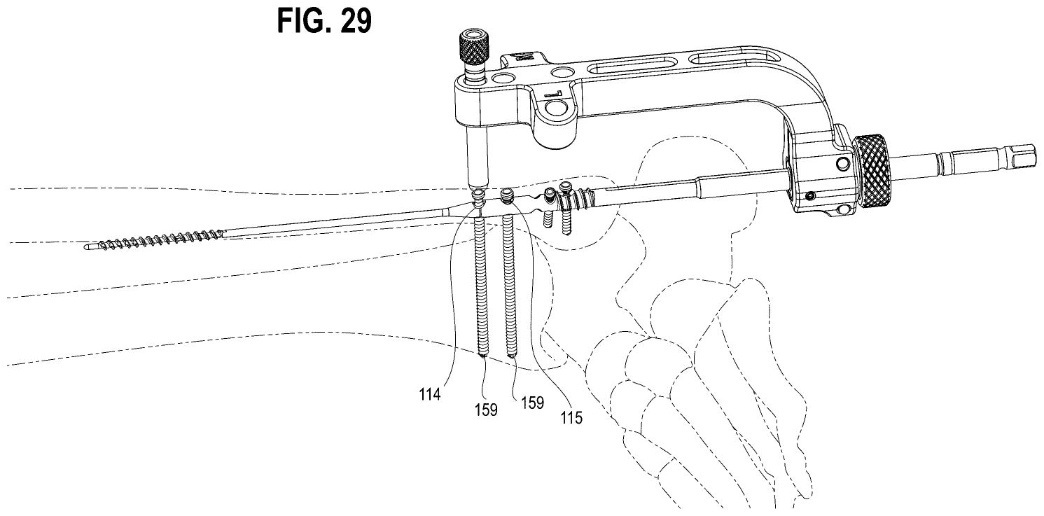

With reference now to FIGS. 25 and 26, once the nail is correctly oriented within the patient's bone, the drill sleeve 170 and screw guide sleeve 171 are then placed into a central hole 153 of the jig. Using the drill (not shown), the surgeon drills a pilot hole into the patient's bone using the drill sleeve 100 to position the drill. As seen in FIG. 26, the drill guide sleeve then is removed and the screw is positioned using the remaining screw guide sleeve 121. The jig is positioned to align the screw guide sleeve with hole 111 in the nail 100. An additional hole is drilled and screw is placed into hole 112 in a similar matter, as seen with respect to FIGS. 27 and 28. With reference to FIG. 29, subsequently, if desired, syndesmotic screws 159 or other devices then may be placed using holes 114, 115 in the jig in a similar manner. This is an exemplary order of placement and in practice the screws or other fastening devices may be placed in a different order.

The drill sleeve 170 and the screw guide sleeve 171 are shown as interfacing concentrically but in an alternative embodiment the drill sleeve and the screw guide sleeve need not interface. An alternative drill sleeve (not shown) may be inserted into a central hole 153 of the jig and the surgeon may drill a pilot hole into the patient's bone. The alternative drill guide sleeve then may be removed, and the alternative screw guide sleeve (not shown) may be positioned into the central hole of the jig. The screw then mat placed through the screw guide sleeve into the patient's bone and through the device.

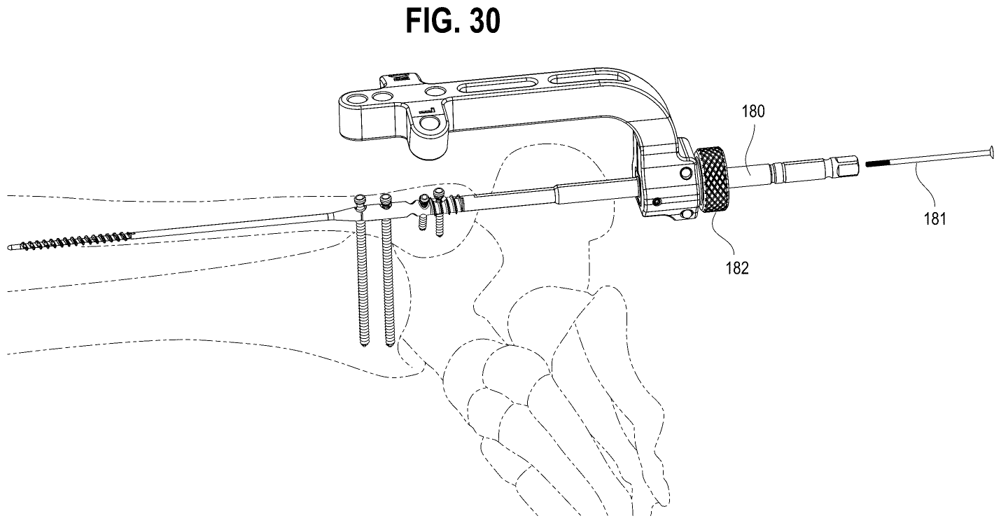

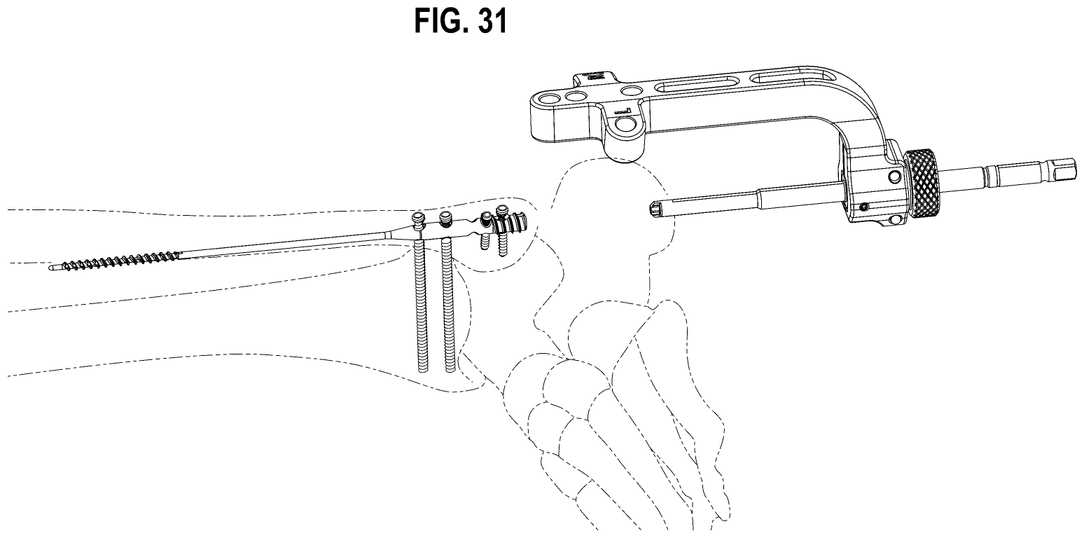

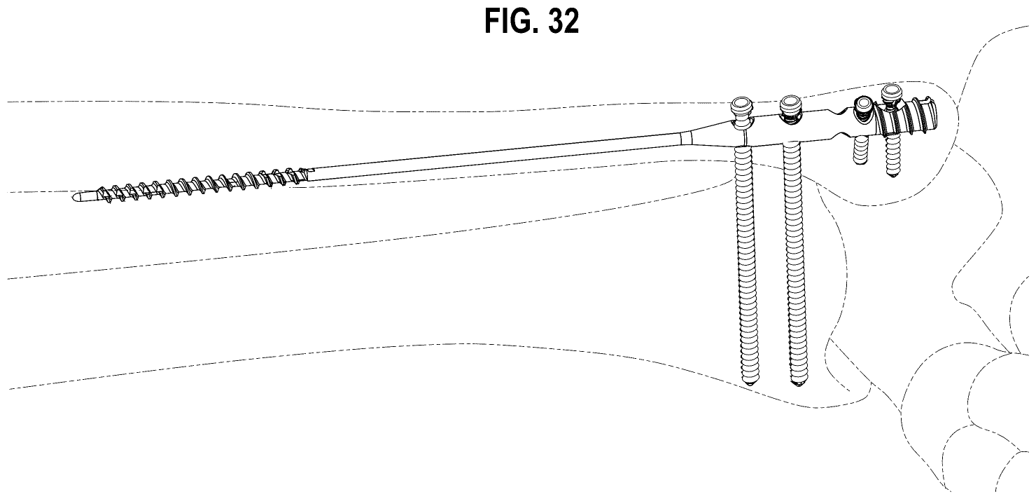

Subsequently, as seen in FIG. 30, the connecting rod 181 is removed and withdrawn from the driver 180. The locking knob 182 then is loosened to remove the drive assembly, as seen in FIG. 31. These steps may be performed in any suitable order. Optionally, an end cap (not shown) may be placed at the head of the nail, if desired. The patient will be left with a secured intramedullary nail, as seen in FIG. 32.

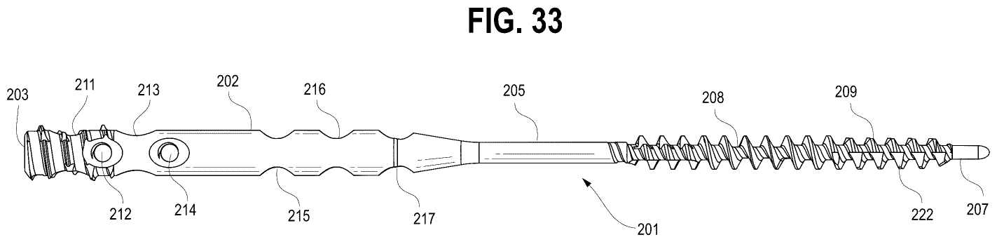

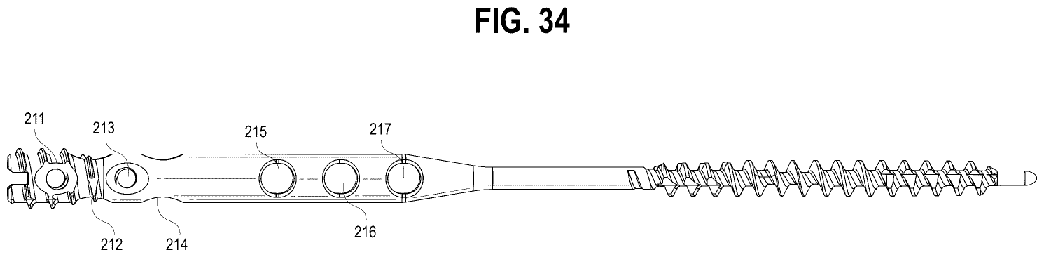

As seen in FIGS. 33 and 34, the alternative intramedullary nail 201 includes transverse through-holes 211, 212, 213, 214, 215, 216, and 217. This includes a first set of holes 215, 216, 217 positioned for syndesmotic securement, and a second set of holes 211, 212, 213, and 214 positioned for transverse non-syndesmotic fibular securement. It is contemplated that not all holes will be used in placement of the nail 201, but the inclusion of seven holes as depicted is believed to afford the surgeon flexibility in the placement of securement screws or other securement devices. Each of the first set of holes 215, 216, 217 is spaced apart along an axis of the intramedullary nail. Also, each the first set of holes has an axis, where the axes of the first set of holes each are generally parallel to one another. Each of the second set of holes 211, 212, 213, and 214 also is spaced apart along an axis of the intramedullary nail. At least one hole (in this case, holes 211, 213) of the second set of holes 211-14 has an axis that is generally parallel to the axes of the first set of holes. Also, at least one hole of the second set of holes (in this case, both holes 212 and 214) has an axis that is not generally parallel to the axes of the first set of holes. Additional or fewer holes may be provided in alternative embodiments.

Like the intramedullary nail 100 discussed above, intramedullary nail 201 includes a proximal portion 202 having an outer surface 203 that is threaded via one or more threads. In the illustrated embodiment, a single thread 204 is included. The intramedullary nail includes a shaft 205 connecting the proximal portion to a distal portion 207, the distal portion comprising a threaded area 208 again comprising one or more threads, one thread 209 in this embodiment.

The thread 209 at the distal portion 207 of the intramedullary nail has a major diameter and a non-uniform minor diameter region. The minor diameter in this embodiment is not uniform in that it varies from a relatively thicker proximal portion, to a thinner region, then to a relatively thicker distal portion. Also, as seen, the thread 209 is interrupted as seen with flat sections 222. Other configurations are possible; for example, the minor diameter may taper from a relatively thicker proximal portion to a relatively thinner distal portion, as per the nail 100.

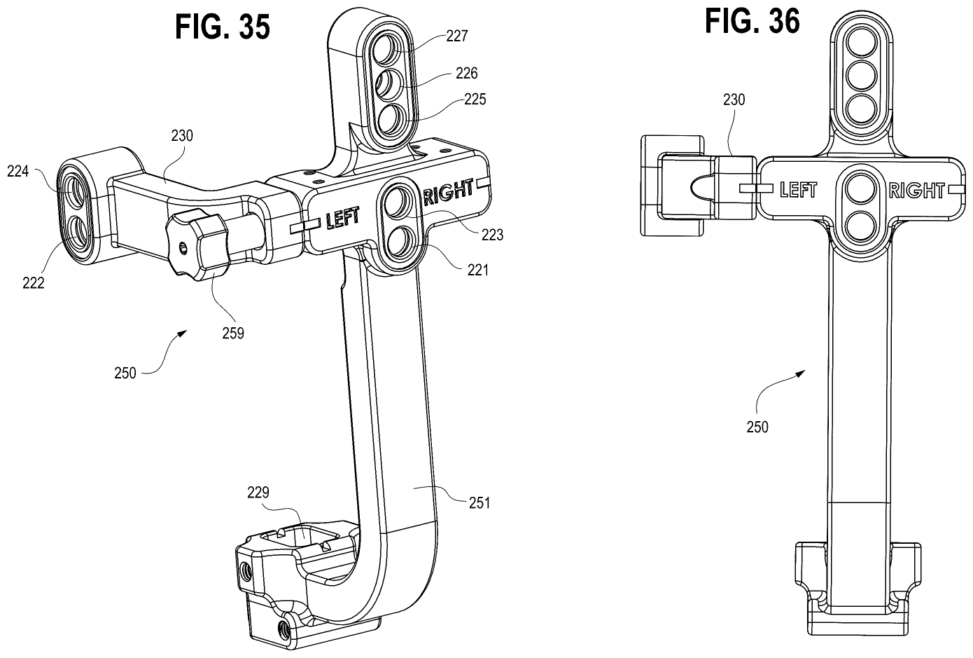

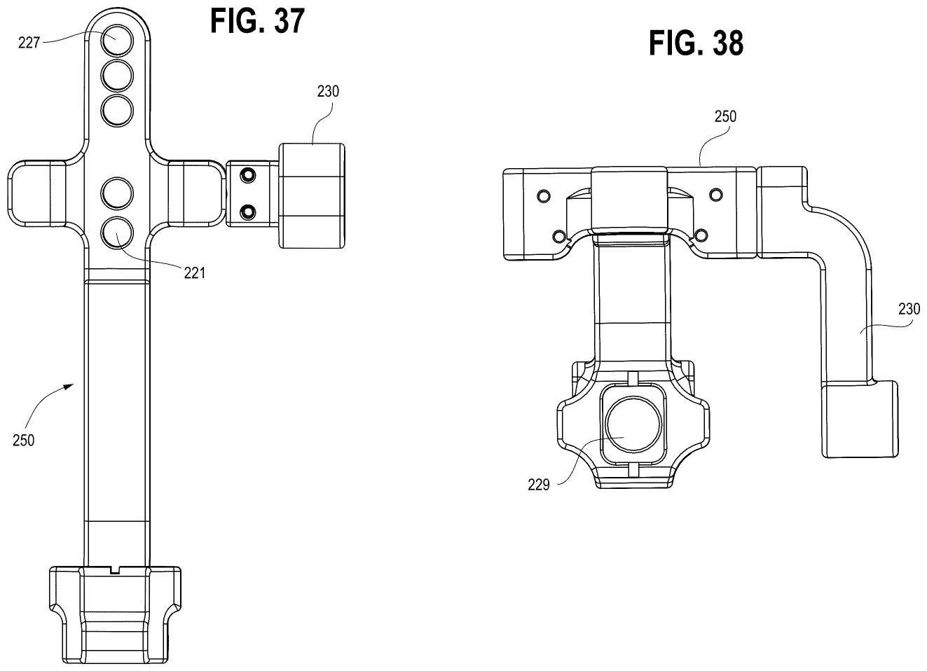

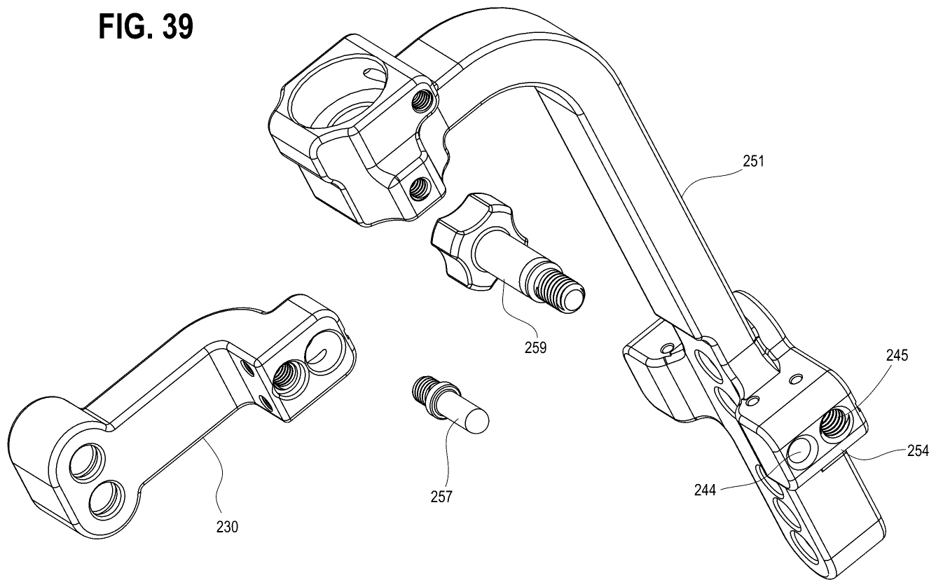

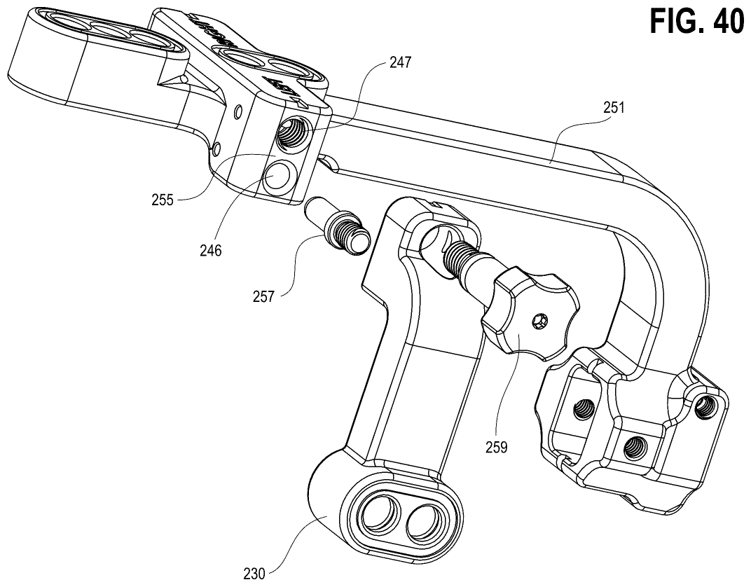

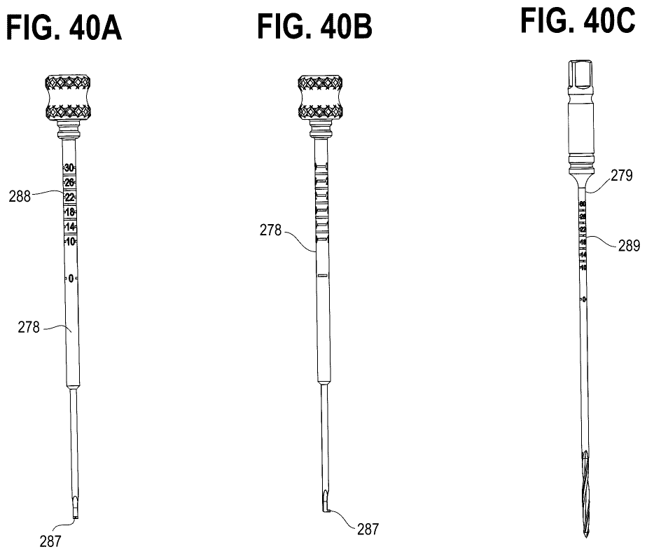

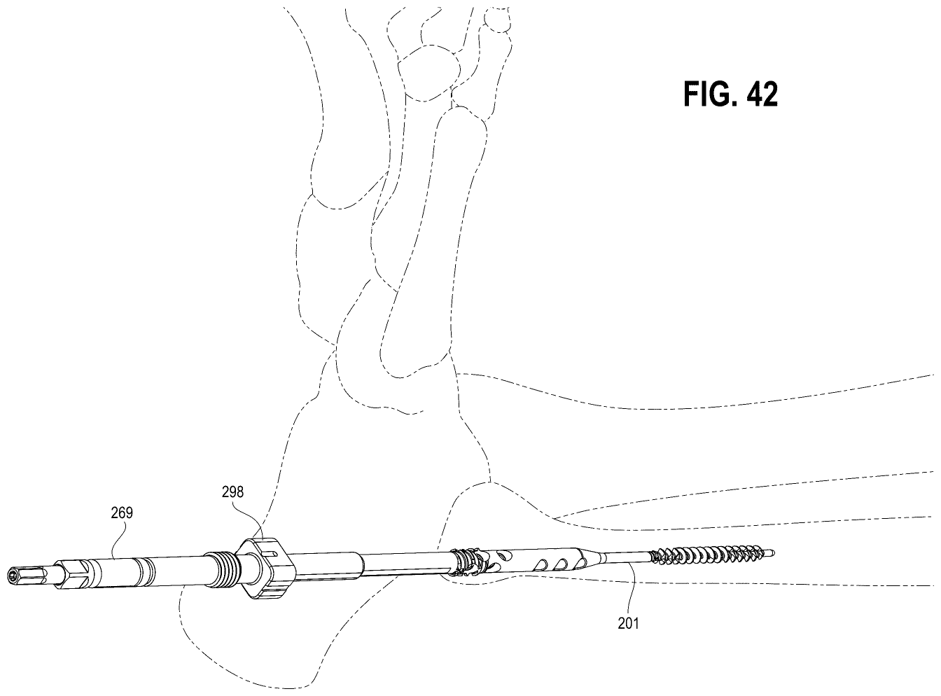

As seen in FIGS. 35-40, the illustrated placement jig 250 includes a jig body 251 having openings 221, 223, 225, 226, and 227, which, when the jig is positioned for use, align respectively with holes 211, 213, 215, 216, and 217 in the nail 201. The placement jig has a keyed driver opening 229 that cooperates with the keyed portion 298 of a driver (seen in FIG. 42) in the manner heretofore described. The jig 250 includes an outrigger section 230 that is releasably connectable to the jig body 251 at a first outrigger connection point 254 (FIG. 39) and a second outrigger connection point 255 (FIG. 40). Each outrigger connection point takes the form of a dual connection system having an unthreaded hole 244 or 246 and a threaded socket 245 or 247 in the illustrated configuration. To connect the outrigger section 230 to the jig body 251, a threaded guidepost 257 is first threaded onto socket 258 (FIG. 39) and then this assembly is secured to the first or second outrigger connection point via threaded knob 259. In the configuration shown in FIG. 40, the knob 259 screws into threaded sockets 245 and the threaded guidepost 257 fits within unthreaded hole 247. The guidepost 257 helps prevent the outrigger section 230 from rotating relative to the jig body 251.

The outrigger section 230 has openings 222, 224 that are intended for alignment respectively with holes 212, 214 in the nail 201. Generally, when the keyed portion of a driver is positioned within the keyed driver opening 229 and when the driver is in engagement with the nail 201, and when the outrigger is connected to the first outrigger connection point, the outrigger section has at least one opening positioned for alignment with a respective hole in the intramedullary nail. Similarly, when the keyed portion of the driver is positioned within the keyed driver opening 229, when the driver is in engagement with the nail, and when the outrigger section is connected to the second outrigger connection point, the outrigger section has at least one opening positioned for alignment with a respective hole in the intramedullary nail. In the illustrated embodiment, the outrigger is configured such that openings 222, 224 are positioned for alignment with holes 212, 214 when attached to either outrigger connection point, i.e., for the left fibula or for the right fibula. In other embodiments the outrigger section may have an opening that aligns with a specific hole in the nail only when the outrigger section is attached to one of the connection points. The jig 250 may be otherwise as configured with respect to jig 150 discussed above.

The intramedullary nail is implanted into a patient for repair of a fibula fracture or other condition of the fibula. The jig 250 is useful in connection with the screw depth gauge 278 shown in FIGS. 40A and 40B and the drill member 279 shown in FIG. 40C. The screw depth gauge has graduation marks 288, visible in FIG. 40A, and terminates in a hook end 287. The drill member 279 also includes graduation marks 289. The graduation marks are useful in determining the size of screw needed for securement of the nail, as described below.

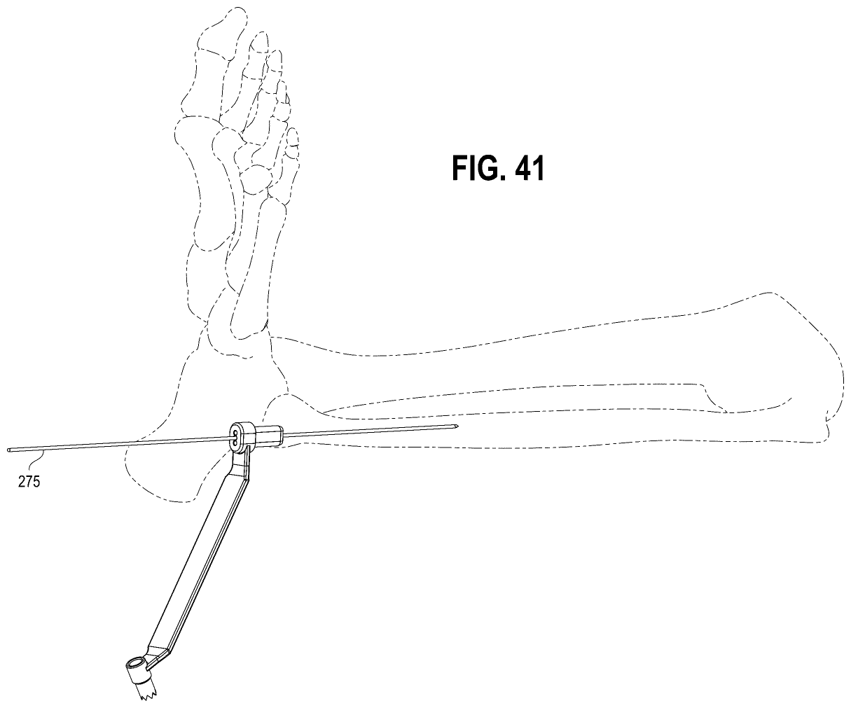

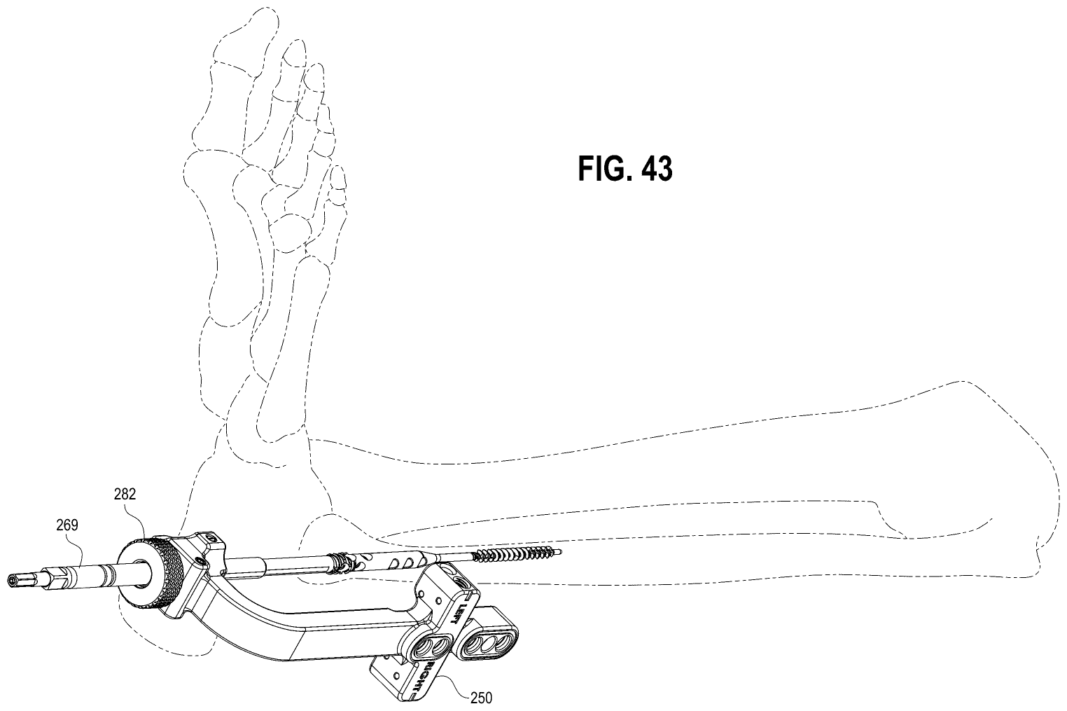

As seen in FIG. 41, the surgeon first uses a guide wire 275 to create a path for an intermedullary channel. After a reaming step (not shown), as seen in FIG. 42, the surgeon connects a drive assembly 269 to the nail 201 and drives the nail into the intermedullary cavity. The user then places the jig 250 over the drive assembly 269 such that the keyed portion 298 of the driver engages and rests in the keyed driver opening 229, resulting in the configuration seen in FIG. 43. The user then secures the jig 250 to the drive assembly 269 with locking knob 282, which engages threaded portion 295 of the driver 280 of the drive assembly 269.

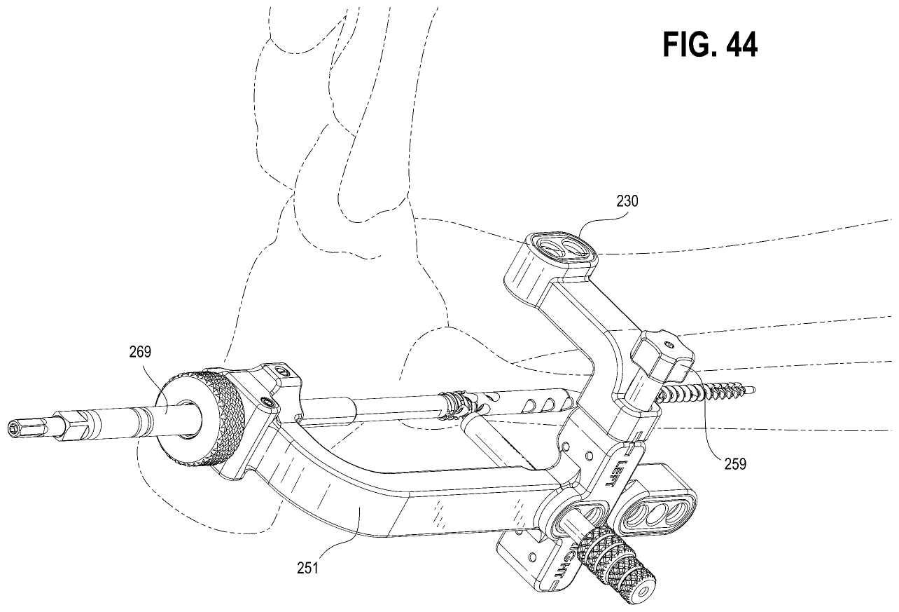

As seen in FIG. 44, the outrigger section 230 is then secured to the body 251 of the jig 250 with knob 259. Alternatively, the outrigger section 230 may be secured to the body 251 prior to placement of the jig 250 onto the drive assembly 269.

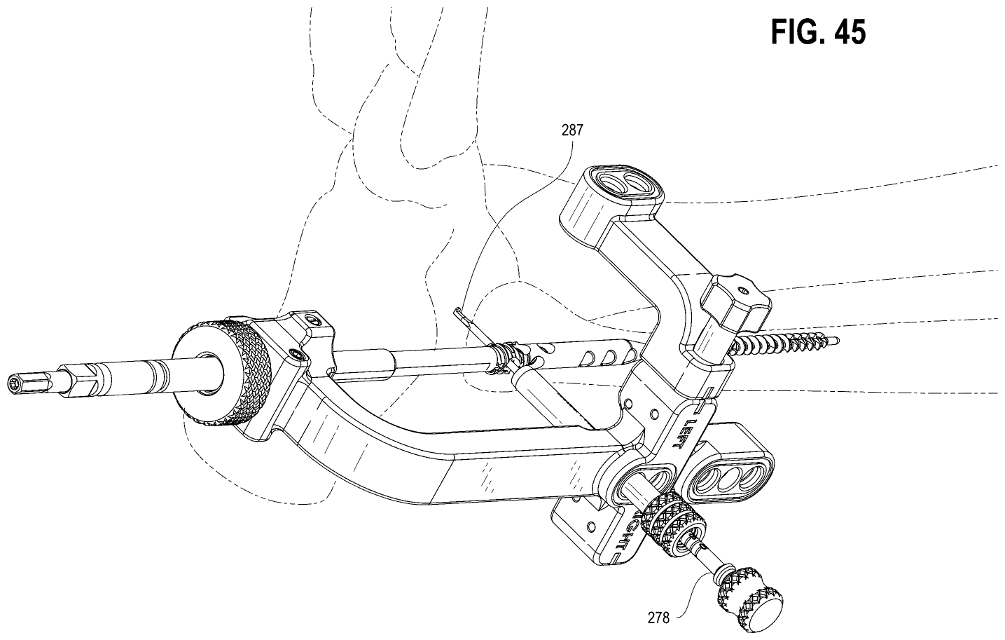

Using radiographic techniques, the surgeon then rotationally positions the nail to the desired position. With the aid of the drill sleeve shown in FIG. 44, the surgeon drills a pilot hole into the patient's bone. As seen in FIG. 45, the drill sleeve then is removed and optionally the drill depth gauge 278 is inserted into the remaining screw guide. The hook 287 is hooked around a distal region of the cortical bone of the fibula, and, using the graduation marks 288 (seen in FIG. 40A) the surgeon is given an indication as to the size of the screw required. Optionally, when drilling a pilot hole using drill member 279, the graduation marks 289 on the drill member may be used for this purpose.

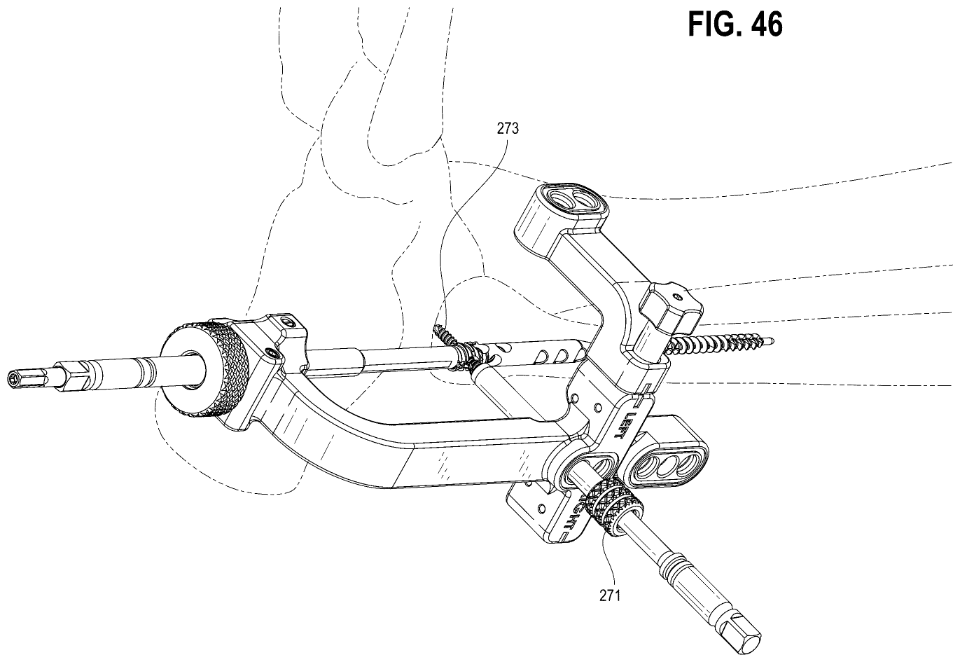

Next, using the screw guide sleeve 271 (FIG. 46), a transverse securement screw 273 or other securement device is then placed into some or all of holes 211, 213, 215, 216, or 217 of the nail 201. It is seen that the openings 221, 223, 225, 227, and 227 serve to align and register the securement devices with respect to the holes in the nail.

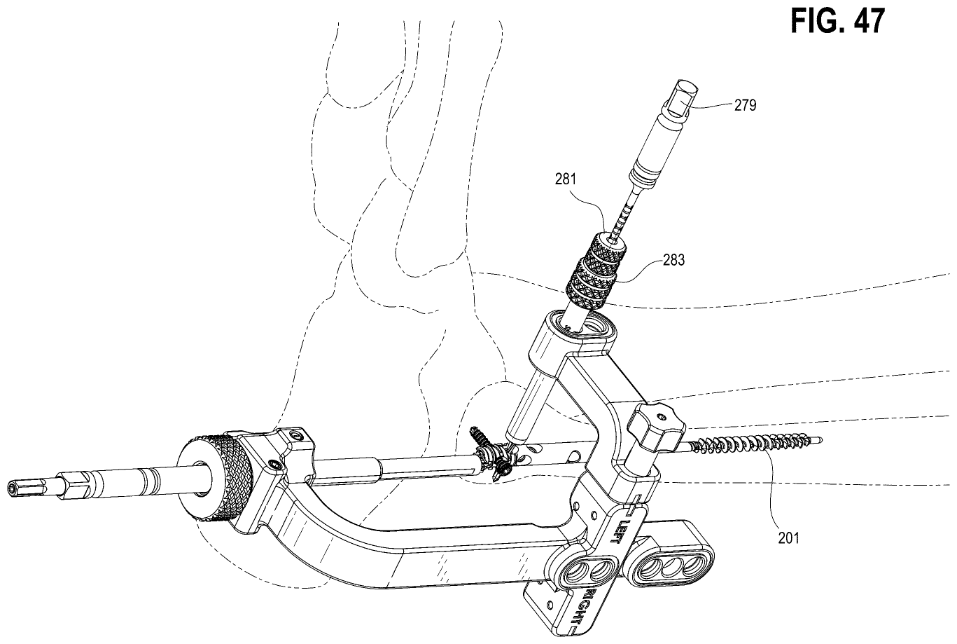

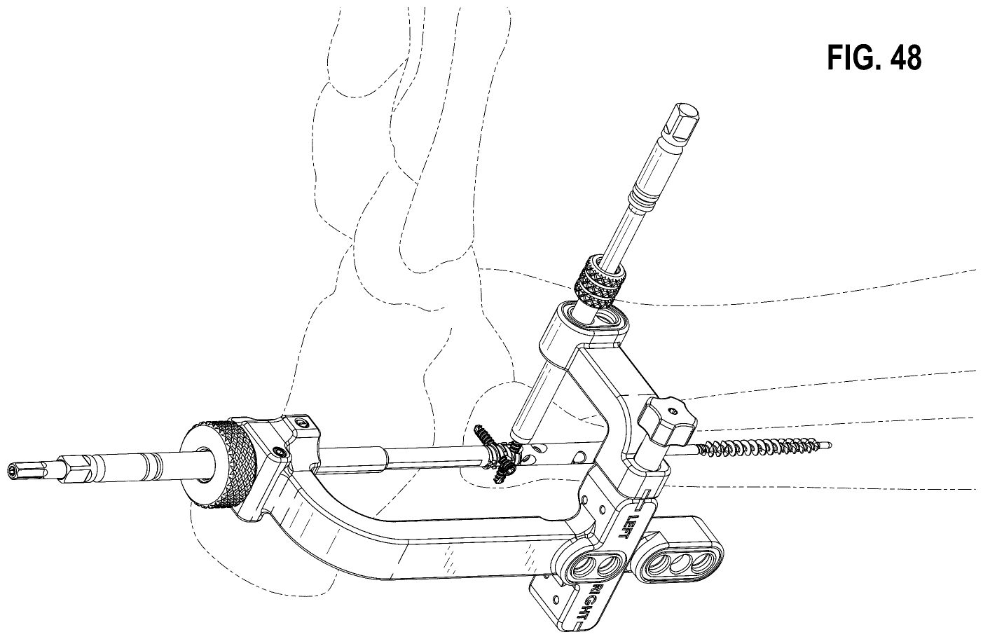

As seen in FIGS. 47 and 48, using one or more openings in the outrigger section 230, this process may be repeated to drill pilot holes and place securement screws into one or both of holes 212, 214 with the aid of drill guide sleeve 281 and screw guide sleeve 283. Again, the openings in the outrigger section serve to align and register the securement devices relative to holes in the nail 201. As before, the sequence of placement of screws or other securement devices may be varied, and screws may be placed via the outrigger section before placing screws via the jig body.

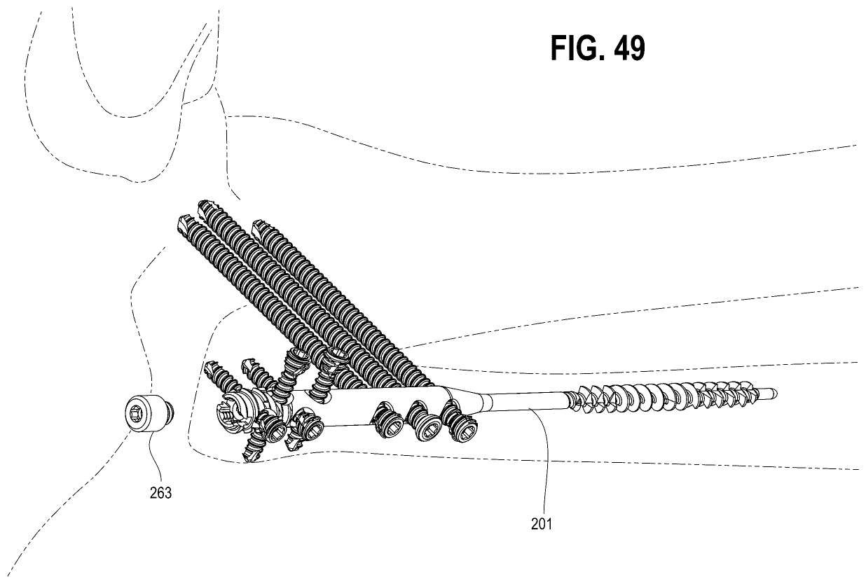

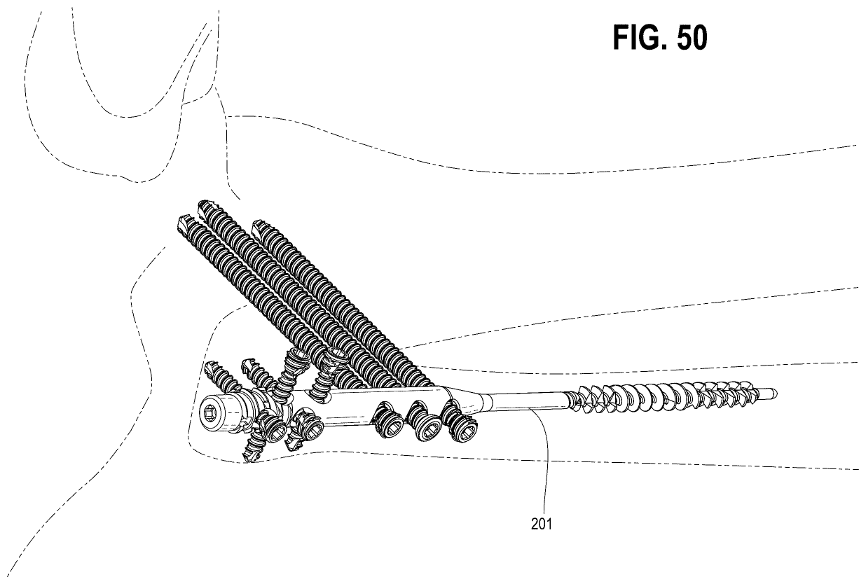

As seen in FIGS. 49 and 50, after placement of at least one and preferably more than one screw, the jig and driver assembly are removed. If desired, an end cap 263 optionally may be screwed into the nail 201. The surgical installation of the nail 201 in any case then will be complete as seen in FIG. 50. FIG. 50 depicts the nail secured with seven screws, but in practice not all seven screws are needed and any one, two, three, four, five, or six screws or other securement devices may be used. The operation then may be closed via conventional techniques.

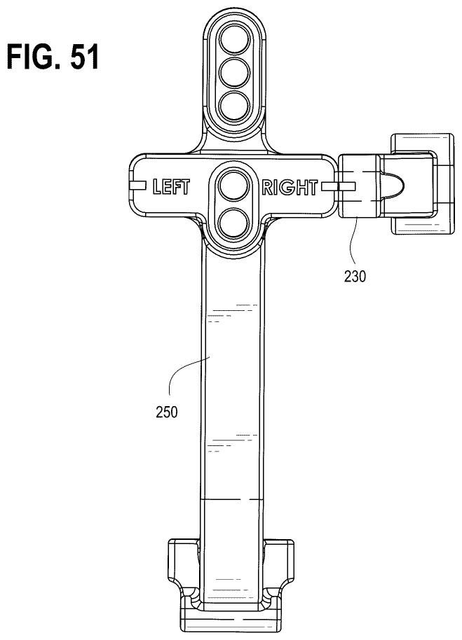

The procedure illustrated herein is shown for a left fibula repair. For a right fibula repair, the procedure is similar except that the outrigger section 230 of the jig 250 should be positioned accordingly as shown in FIG. 51.

The method is described with reference to a surgeon, which in practice is intended to encompass plural members of a surgical team. The intramedullary nail described herein may be used for fibular fracture repair but is not limited to such uses.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. An intramedullary nail comprising a proximal portion, a shaft, and a distal portion, both a proximal portion and the distal portion being threaded and the intramedullary nail including plural transverse holes.

2. The intramedullary nail of claim 1, the distal threaded portion being tapered.

3. The intramedullary nail of claim 1, the distal portion having only a portion of which being tapered and having other portions not tapered.

4. The intramedullary nail of claim 1, the distal portion having a staggered configuration.

5. The intramedullary nail of claim 1, having an interrupted distal thread.

6. The intramedullary nail of claim 1, including a first set of holes positioned for syndesmotic securement and a second set of holes positioned for distal securement, each of the first set of holes being spaced apart along an axis of the intramedullary nail, each of the first set of holes having an axis, the axes of the first set of holes being generally parallel to one another, each of the second set of holes being spaced apart along an axis of the intramedullary nail and the second set of holes being disposed relatively proximally to the second set of holes.

7. The intramedullary nail of claim 6, at least one hole of the second set of holes having an axis that is generally parallel to the axes of the first set of holes and at least one hole of the second set of holes having an axis that is not generally parallel to the axes of the first set of holes.

8. A kit comprising the intramedullary nail of claim 1, a driver, and a placement jig, the placement jig comprising a keyed driver opening and a plurality of holes, the driver comprising a keyed portion that may be accommodated within the keyed driver opening and engageable with said intramedullary nail, and the placement jig further comprising a plurality of openings, at least some of the openings positioned for alignment with respective holes in said intramedullary nail when said keyed portion of said driver is positioned within said keyed driver opening and when said driver is in engagement with said nail.

9. A kit according to claim 8, the placement jig having a body including a first outrigger connection point and a second outrigger connection point, the jig including an outrigger section, the outrigger section being selectively connectable to the body at the first outrigger connection point or the second outrigger connection point, the outrigger section having at least one opening positioned for alignment with a respective hole in said intramedullary nail when said keyed portion of said driver is positioned within said keyed driver opening and when said driver is in engagement with said nail and when said outrigger section is connected to the first outrigger connection point, the outrigger section having at least one opening positioned for alignment with a respective hole in said intramedullary nail when said keyed portion of said driver is positioned within said keyed driver opening and when said driver is in engagement with said nail and when said outrigger section is connected to the second outrigger connection point.

10. A kit according to claim 8 and a transverse screw.

11. A fibula repair method comprising:
providing an intramedullary nail, said intramedullary nail comprising a proximal portion, a shaft, and a distal portion, both a proximal portion and the distal portion being threaded and the intramedullary nail including plural transverse holes;
driving said intramedullary nail into an intramedullary fibular cavity of a patient whereby both proximal and distal portions of said intramedullary nail are threadingly engaged with bone; and
securing said intramedullary nail by placing a securement device through one of the transverse holes.

12. The fibula repair method of claim 11, the securement device comprising a screw.

13. The fibula repair method of claim 11, including placing a syndesmotic securement device and a second securement device.

14. The fibula repair method of claim 11, said intramedullary nail including a first set of holes positioned for syndesmotic securement and a second set of holes positioned for distal securement, each of the first set of holes being spaced apart along an axis of the intramedullary nail, each of the first set of holes having an axis, the axes of the first set of holes being generally parallel to one another, each of the second set of holes being spaced apart along an axis of the intramedullary nail and the second set of holes being disposed relatively proximally to the second set of holes, the method comprising placing a securement device through a hole of the first set of holes and placing a securement device through a hole of the second set of holes.

15. The fibula repair method of claim 11, the driver including a keyed portion, the method including
providing a placement jig comprising a keyed driver opening and a plurality of holes, the driver comprising a keyed portion that may be accommodated within the keyed driver opening and engageable with said intramedullary nail, and the placement jig further comprising a plurality of openings, at least some of the openings positioned for alignment with respective holes in said intramedullary nail when said keyed portion of said driver is positioned within said keyed driver opening and when said driver is in engagement with said nail;
positioning said placement jig over said driver, the placement jig having a keyed driver opening and the keyed portion of said driver positioned within said keyed driver opening; and
placing a securement device through a hole of the first set of holes using an aligned hole of the placement jig and placing a securement device through a hole of the second set of holes using an aligned hole of the placement jig.

16. The fibula repair method of claim 15, the placement jig having a body including a first outrigger connection point and a second outrigger connection point, the jig including an outrigger section, the outrigger section being connected to the body at the first outrigger connection point or the second outrigger connection point, the outrigger having at least one opening positioned for alignment with a respective hole in said intramedullary nail when said keyed portion of said driver is positioned within said keyed driver opening and when said driver is in engagement with said nail and when said outrigger section is connected to the first outrigger connection point, the outrigger section having at least one opening positioned for alignment with a respective hole in said intramedullary nail when said keyed portion of said driver is positioned within said keyed driver opening and when said driver is in engagement with said nail and when said outrigger section is connected to the second outrigger connection point, the method including placing a securement device through a hole of the first set of holes using an aligned hole of the outrigger section of the placement jig.

17. The fibula repair method of claim 11, said intramedullary nail having an interrupted distal thread.
